(19) United States
(12) Patent Application Publication
(10) Pub. No.: US 2008/0286875 A1
(43) Pub. Date: Nov. 20, 2008

(54) RYR2 MUTATIONS

(76) Inventors: Michael J. Ackerman, Rochester, MN (US); David J. Tester, Rochester, MN (US); Melissa L. Will, Dodge Center, MN (US); Carla M. Haglund, Pine Island, MN (US)

Correspondence Address: FISH & RICHARDSON P.C., PO BOX 1022, MINNEAPOLIS, MN, 55440-1022, US

(21) Appl. No.: 11/741,628

(22) Filed: Apr. 27, 2007

Related U.S. Application Data
(60) Provisional application No. 60/796,175, filed on Apr. 28, 2006.

(51) Int. Cl. G01N 33/00

(52) U.S. Cl. 436/86; 436/94

(57) ABSTRACT

This document provides methods and materials related to assessing a mammal for the presence or absence of a genetic mutation. For example, methods for determining whether or not a mammal contains a genetic mutation in an RyR2 sequence are provided. In addition, isolated nucleic acid molecules containing an RyR2 sequence and encoding a mutation are provided herein.

### CROSS-REFERENCE TO RELATED APPLICATIONS

This application claims benefit of priority under 35 U.S.C. §119(e) to U.S. provisional application Ser. No. 60/796,175, filed Apr. 28, 2006.

### STATEMENT AS TO FEDERALLY SPONSORED RESEARCH

This invention was made with government support under HD042569 awarded by National Institutes of Health. The government has certain rights in the invention.

### BACKGROUND

1. Technical Field

This document relates to methods and materials involved in assessing mammals (e.g., humans) for RyR2 mutations.

2. Background Information

Congenital long QT syndrome (LQTS) is the prototypic cardiac channelopathy characterized by delayed repolarization of the myocardium, QT prolongation, and increased clinical risk for syncope, seizures, and sudden cardiac death (Vincent et al., N. Engl. J. Med., 327:846-852 (1992); Moss and Robinson, Ann. N.Y. Acad. Sci., 644:103-111 (992); and Ackerman, Mayo Clin. Proc., 73:250-269 (1998)). To date, hundreds of LQTS-causing mutations have been discovered and about 75 percent of clinically robust LQTS can be genetically elucidated with pathogenic mutations identifiable in three genes encoding critical ion channel sub-units, (Splawski et al., Circulation, 102:1178-1185 (2000) and Tester et al., Heart Rhythm, 2:507-517 (2005)): KCNQ1/KVLQT1 (LQT1; Wang et al., Nat. Genet., 12:17-23 (1996)), KCNH2/HERG (LQT2; Curran et al., Cell, 80:795-803 (1995)), SCN5A (LQT3; Wang et al., Cell, 80:805-811 (1995)).

Compared to LQTS, catecholaminergic polymorphic ventricular tachycardia (CPVT) is a more recent addition to the compendium of cardiac channelopathies (Leenhardt et al., Circulation, 91:1512-1519 (1995)). Genetically, CPVT1-associated mutations, residing in critical regions of the RyR2-encoded cardiac ryanodine receptor/calcium release channel, account for about 50 to 65 percent of CPVT, whereas a small minority of patients has type 2 CPVT secondary to mutations in CASQ2-encoded calsequestrin (Lahat et al., Circulation, 103:2822-2827 (2001); Priori et al., Circulation, 103:196-200 (2001); Priori et al., Circulation, 106:69-74 (2002); Marks, Circulation, 106(1):8-10 (2002); Lahat et al., Circulation, 107(3):e29 (2003); and Postma et al., Circulation Research, 91:E21-E26 (2002)). Phenotypically, CPVT is characterized by exertional syncope or sudden death in a structurally normal heart. The resting electrocardiogram in CPVT is completely normal. The electrocardiographic signature of CPVT is either exercise- or catecholamine-induced ventricular dysrhythmia. CPVT1 closely mimics the phenotype of LQTS, particularly concealed LQT1.

### SUMMARY

This document provides methods and materials related to assessing a mammal for the presence or absence of a genetic mutation. For example, this document provides methods for determining whether or not a mammal contains a genetic mutation in an RyR2 sequence. The presence of a genetic mutation in an RyR2 sequence can indicate that the mammal has an increased susceptibility of having syncope, a seizure, sudden infant death syndrome (SIDS), or a cardiac event. In some cases, the presence of a genetic mutation in an RyR2 sequence can indicate that the mammal has LQTS or CPVT. Assessing a mammal for the presence or absence of a genetic mutation in an RyR2 sequence can help clinicians determine appropriate treatment options. For example, a mammal identified as having a mutation provided herein can be determined to have CPVT and can be treated with a calcium channel blocker/beta blocker and/or receive an implantation of an internal cardioverter-defibrillator.

This document also provides isolated nucleic acid molecules having a portion of an RyR2 sequence suspected to contain one or more of the mutations provided herein. Such nucleic acid molecules can be sequenced to determine whether or not the mammal contains a mutation provided herein. Using the nucleic acid molecules provided herein to assess a mammal for the presence or absence of a genetic mutation in an RyR2 sequence can help clinicians determine appropriate treatment options.

In general, one aspect of this document features a method for determining whether or not a human contains a genetic mutation associated with an increased susceptibility of having syncope, a seizure, or a cardiac event. The method comprises, or consists essentially of, determining whether or not nucleic acid from the human encodes a mutation at an amino acid position of SEQ ID NO:1 identified on a list, wherein the list is on a tangible medium and comprises amino acid position 164, 176, 186, 243, 329, 332, 357, 400, 414, 419, 420, 466, 919, 1724, 1837, 2113, 2246, 2267, 2387, 2392, 2403, 2420, 2475, 3800, 3938, 4097, 4124, 4146, 4158, 4196, 4307, 4497, 4499, 4510, 4556, 4565, 4657, 4658, 4671, 4848, 4887, 4936, or 4959 of SEQ ID NO:1 (or any combination thereof). The list that is on a tangible medium can comprise amino acid position 186, 243, 329, 332, 357, 414, 466, 919, 1724, 1837, 2387, 2420, 2475, 3800, 3938, 4124, 4196, 4307, 4556, 4657, 4658 or 4887 of SEQ ID NO:1 (or any combination thereof). The cardiac event can be sudden cardiac death. The nucleic acid can comprise an exon of a human RyR2 gene. The nucleic acid can comprise two or more exons of a human RyR2 gene. The list can identify a single amino acid position of SEQ ID NO:1. The list can comprise at least two amino acid positions selected from the group consisting of amino acid positions 186, 243, 329, 332, 357, 414, 466, 919, 1724, 1837, 2387, 2420, 2475, 3800, 3938, 4124, 4196, 4307, 4556, 4657, 4658, and 4887 of SEQ ID NO:1. The list can comprise amino acid positions 186, 243, 329, 332, 357, 414, 466, 919, 1724, 1837, 2387, 2420, 2475, 3800, 3938, 4124, 4196, 4307, 4556, 4657, 4658, and 4887 of SEQ ID NO:1. The tangible medium can comprise a paper medium. The tangible medium can comprise a computer medium. The tangible medium can comprise a computer medium, and the list can be contained within a database stored on the computer medium. The method can comprise reporting the presence or absence of the mutation to a medical professional or to the human. The method can comprise classifying the human as having an increased susceptibility of having syncope, a seizure, or a cardiac event based on the presence of the mutation. The method can comprise classifying the human as having type 1 catecholaminergic polymorphic ventricular tachycardia. The method can comprise classifying the human as having long QT syndrome. The human can experience a cardiac event prior to the determining step. The human can experience a drowning event prior to the determining step. The method can be performed postmortem.

In another aspect, this document features a method for determining whether or not a human contains a genetic mutation associated with an increased susceptibility of having sudden infant death syndrome or sudden unexplained death syndrome. The method comprises, or consists essentially of, determining whether or not nucleic acid from the human encodes a mutation at an amino acid position of SEQ ID NO:1 identified on a list, wherein the list is on a tangible medium and comprises amino acid position 164, 176, 186, 243, 329, 332, 357, 400, 414, 419, 420, 466, 919, 1724, 1837, 2113, 2246, 2267, 2387, 2392, 2403, 2420, 2475, 3800, 3938, 4097, 4124, 4146, 4158, 4196, 4307, 4497, 4499, 4510, 4556, 4565, 4657, 4658, 4671, 4848, 4887, 4936, or 4959 of SEQ ID NO:1 (or any combination thereof). The list that is on a tangible medium can comprise amino acid position 400, 2113, 2267, 2392, 4565, or 4936 of SEQ ID NO:1 (or any combination thereof). The nucleic acid can comprise an exon of a human RyR2 gene. The nucleic acid can comprise two or more exons of a human RyR2 gene. The list can identify a single amino acid position of SEQ ID NO:1. The list can comprise amino acid positions 400, 2113, 2267, 2392, 4565, or 4936 of SEQ ID NO:1. The tangible medium can comprise a paper medium. The tangible medium can comprise a computer medium. The tangible medium can comprise a computer medium, and the list can be contained within a database stored on the computer medium. The method can comprise reporting the presence or absence of the mutation to a medical professional or to the human. The method can comprise classifying the human as having an increased susceptibility of having sudden infant death syndrome or sudden unexplained death syndrome based on the presence of the mutation. The human can experience increased sympathetic activity prior to the determining step. The method can be performed postmortem.

In another aspect, this document features an isolated nucleic acid molecule. The isolated nucleic acid molecule can comprise, or consist essentially of, a nucleic acid sequence encoding a portion of the amino acid sequence set forth in SEQ ID NO:1, wherein the nucleic acid molecule comprises a sequence that encodes an amino acid mutation at position 164, 176, 186, 243, 329, 332, 357, 400, 414, 419, 420, 466, 919, 1724, 1837, 2113, 2246, 2267, 2387, 2392, 2403, 2420, 2475, 3800, 3938, 4097, 4124, 4146, 4158, 4196, 4307, 4497, 4499, 4510, 4556, 4565, 4657, 4658, 4671, 4848, 4887, 4936, or 4959 of SEQ ID NO:1 (or any combination thereof). The nucleic acid molecule can comprise a sequence that encodes an amino acid mutation at position 186, 243, 329, 332, 357, 400, 414, 466, 919, 1724, 1837, 2113, 2267, 2387, 2392, 2420, 2475, 3800, 3938, 4124, 4196, 4307, 4556, 4565, 4657, 4658, 4887, or 4936 of SEQ ID NO:1 (or any combination thereof). The portion can comprise at least five contiguous amino acid residues set forth in SEQ ID NO:1. The portion can comprise at least ten contiguous amino acid residues set forth in SEQ ID NO:1. The nucleic acid molecule can comprise the portion followed by the sequence followed by a second portion of the amino acid sequence set forth in SEQ ID NO:1, wherein the portion and the second portion each comprise at least five contiguous amino acid residues set forth in SEQ ID NO:1.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a diagram of the predicted linear channel topology of RYR2 (not drawn to scale) with CPVT1-associated variants. The approximate locations of pathogenic CPVT1-causing mutations are indicated. The black box represents the calstabin-2 binding domain (amino acids 2361-2496). The number within the circle corresponds to the case # on Tables 2 and 3.

FIG. 2 is a bar graph plotting the relative yields by QTc for cases presenting with exertion-induced events. The percentage (%) of genotype are shown for the indicated QTc (ms) ranges. For example, 44% ( 4/9) of RyR2-positive cases with exertion-induced events and an available QTc, had a QTc<420 ms.

FIG. 3 contains chromatograms of heteroduplexes containing the R2267H (panel A) and S4565R (panel B) mutations analyzed by denaturing high performance liquid chromatography. FIG. 3 also contains direct DNA sequence chromatograms for the R2267H (panel C) and S4565R (panel D) mutations. FIG. 3E is a schematic diagram of the predicted linear RyR2 channel topology (not drawn to scale) with localization of the R2267H and S4565R mutations indicated by white circles. The black bar represents the calstabin-2 binding domain.

FIG. 4 contains representative single channel current traces of untreated and PKA phosphorylated RyR2-WT (panels A and B) and RyR2-R2267H (panels C and D) channels at two different calcium concentrations, as indicated to the left of the traces (150 nM first raw, 700 nM second raw). Channel openings are shown as upward deflections; the open and closed (c) states of the channel are indicated by horizontal bars at the beginning of each trace, and the corresponding amplitude histograms are on the right side of each current trace. An example of channel activity is shown at two different time scales (5 s for upper trace; 500 ms for lower trace) as indicated by the dimension bars, and the Po (open probability), To (average open time) and Tc (average closed time) are indicated above each trace. FIG. 4E is a graph plotting the open probability of mutant and WT channels at Ca²⁺ concentrations ranging from 90 nM to 2 μM (full activation). Each data point represents the open probability calculated as an average of several independent experiments shown as mean ±SE. Ca²⁺ dependences of untreated RyR2-WT (open triangles, dashed line, n=4), RyR2-R2267H (full triangles, solid line, n=7) and PKA phosphorylated RyR2-WT (open squares, dotted line, n=4), RyR2-R2267H (full circles, solid line, n=3) were fitted to a sigmoidal equation. FIG. 4F contains immunoblots analyzing polypeptides immunoprecipitated from untreated and PKA phosphorylated RyR2-WT and RyR2-R2267H microsomes with an anti-RyR2 antibody. The immunoblots were developed using anti-RyR2, anti-calstabin, or phospho-specific RyR2 antibodies.

FIG. 5 is a listing of an amino acid sequence (SEQ ID NO:1) of a human RyR2 polypeptide.

### DETAILED DESCRIPTION

This document provides methods and materials related to assessing a mammal for the presence or absence of a genetic mutation. For example, this document provides methods for determining whether or not a mammal contains a genetic mutation in an RyR2 sequence. As described herein, a mammal having one or more than one mutation in an RyR2 sequence disclosed herein (e.g., one or more than one mutation set forth in Table 1) can be identified as being susceptible to having SIDS, SUDS, syncope, a seizure, or a cardiac event.

Examples of RyR2 sequences include human RyR2 polypeptide and nucleic acid sequences. A human RyR2 polypeptide can have the amino acid sequence set forth in GenBank accession number NM_{—}001035.1 (GI:4506756) and can be encoded by a nucleic acid sequence set forth in GenBank accession number AJ300340 (GI:11878410). Possible RyR2 genetic mutations include, without limitation, those that result in an amino acid insertion, deletion, substitution, or combination thereof at amino acid position 186, 243, 329, 332, 357, 400, 414, 466, 919, 1724, 1837, 2113, 2267, 2387, 2392, 2420, 2475, 3800, 3938, 4124, 4196, 4307, 4556, 4565, 4657, 4658, 4887, and 4936. For example, V186 can be deleted or changed to an amino acid residue other than V, E243 can be deleted or changed to an amino acid residue other than E, F329 can be deleted or changed to an amino acid residue other than F, R332 can be deleted or changed to an amino acid residue other than R, G357 can be deleted or changed to an amino acid residue other than G, D400 can be deleted or changed to an amino acid residue other than D, R414 can be deleted or changed to an amino acid residue other than R, P466 can be deleted or changed to an amino acid residue other than P, V919 can be deleted or changed to an amino acid residue other than V, E1724 can be deleted or changed to an amino acid residue other than E, E1837 can be deleted or changed to an amino acid residue other than E, V2113 can be deleted or changed to an amino acid residue other than V, R2267 can be deleted or changed to an amino acid residue other than R, Y2392 can be deleted or changed to an amino acid residue other than Y, A2387 can be deleted or changed to an amino acid residue other than A (e.g., T), R2420 can be deleted or changed to an amino acid residue other than R, V2475 can be deleted or changed to an amino acid residue other than V, C3800 can be deleted or changed to an amino acid residue other than C, S3938 can be deleted or changed to an amino acid residue other than S, S4124 can be deleted or changed to an amino acid residue other than S, T4196 can be deleted or changed to an amino acid residue other than T, R4307 can be deleted or changed to an amino acid residue other than R, A4556 can be deleted or changed to an amino acid residue other than A, S4565 can be deleted or changed to an amino acid residue other than S, K4887 can be deleted or changed to an amino acid residue other than K, and/or G4936 can be deleted or changed to an amino acid residue other than G. In some cases, a mutation can result in one or more amino acid residues being inserted at a particular location. For example, a mutation can result in two amino acid residues being inserted at amino acid position 4657. Possible nucleic acid mutations include, without limitation, those provided in Table 1, 2, 3, or 6.

TABLE 1
RyR2 genetic mutations.

| Gene | Exon | Base Position | Nucleotide | Variant | Setting | Mutation/Polymorphism |
|---|---|---|---|---|---|---|
| RYR2 | 8 | 490 | 490 C > T | P164S | CPVT | Mutation |
| RYR2 | 8 | 527 | 527 G > A | R176Q | LQTS | Mutation |
| RYR2 | 8 | 556 | 556 G > A | V186M | CPVT | Mutation |
| RYR2 | 10 | 727 | 727 G > A | E243K | LQTS | Mutation |
| RYR2 | 12 | 985 | 985 T > C | F329L | LQTS | Mutation |
| RYR2 | 12 | 994 | 994 C > T | R332W | LQTS | Mutation |
| RYR2 | 13 | 1069 | 1069 G > A | G357S | LQTS | Mutation |
| RYR2 | 14 | 1198 | 1198 G > C | D400H | SUDS | Mutation |
| RYR2 | 14 | 1240 | 1240 C > T | R414C | Drowning | Mutation |
| RYR2 | 14 | 1241 | 1241 G > T | R414L | LQTS | Mutation |
| RYR2 | 14 | 1255 | 1255 A > T | I419F | LQTS | Mutation |
| RYR2 | 14 | 1258 | 1258 C > T | R420W | SUDS* | Mutation |
| RYR2 | 15 | 1396 | 1396 C > G | P466A | LQTS | Mutation |
| RYR2 | 24 | 2755 | 2755 G > A | V919M | HCM** | Mutation |
| RYR2 | 37 | 5170 | 5170 G > A | E1724K | CPVT | Mutation |
| RYR2 | 37 | 5171 | 5171 A > T | E1724V | LQTS | Mutation |
| RYR2 | 37 | 5509 | 5509 G > A | E1837K | LQTS | Mutation |
| RYR2 | 37 | 5654 | 5654 G > A | G1885E | health | Polymorphism |
| RYR2 | 37 | 5656 | 5656 G > A | G1886S | health | Polymorphism |
| RYR2 | 44 | 6337 | 6337 G > A | V2113M | SUDS | Mutation |
| RYR2 | 44 | 6737 | 6737 C > T | S2246L | SUDS | Mutation |
| RYR2 | 45 | 6800 | 6800 G > A | R2267H | SIDS | Mutation |
| RYR2 | 46 | 6962 | 6962 T > C | Y2392C | SUDS | Mutation |
| RYR2 | 47 | 7158 | 7158 G > A | A2387T | LQTS | Mutation |
| RYR2 | 47 | 7165 | 7165 A > C | M2389L | health | Polymorphism |
| RYR2 | 47 | 7175 | 7175 A > G | Y2392C | SUDS | Mutation |
| RYR2 | 47 | 7207 | 7207 G > A | A2403T | LQTS | Mutation |
| RYR2 | 48 | 7258 | 7258 A > T | R2420W | LQTS | Mutation |
| RYR2 | 49 | 7423 | 7423 G > T | V2475F | Drowning | Mutation |
| RYR2 | 61 | 8873 | 8873 A > G | Q2958R | health | Polymorphism |
| RYR2 | 83 | 11399 | 11399 G > T | C3800F | LQTS | Mutation |
| RYR2 | 88 | 11814 | 11814 C > A | S3938R | CPVT | Mutation |
| RYR2 | 90 | 12028 | 12028 A > G | V4010M | health | Polymorphism |
| RYR2 | 90 | 12290 | 12290 A > G | N4097S | SUDS | Mutation |
| RYR2 | 90 | 12371 | 12371 G > C | S4124T | LQTS | Mutation |
| RYR2 | 90 | 12436 | 12436 G > A | E4146K | SUDS | Mutation |
| RYR2 | 90 | 12472 | 12472 A > C | T4158P | SUDS | Mutation |
| RYR2 | 90 | 12586 | 12586 A > G | T4196A | CPVT | Mutation |
| RYR2 | 90 | 12845 | 12845 C > T | A4282V | health | Polymorphism |
| RYR2 | 90 | 12919 | 12919 C > T | R4307C | LQTS | Mutation |
| RYR2 | 93 | 13489 | 13489 C > T | R4497C | SUDS | Mutation |
| RYR2 | 93 | 13496 | 13496 T > G | F4499C | LQTS | Mutation |
| RYR2 | 93 | 13528 | 13528 G > A | A4510T | LQTS | Mutation |
| RYR2 | 94 | 13666 | 13666 G > A | A4556T | LQTS | Mutation |
| RYR2 | 94 | 13695 | 13695 C > A | S4565R | SIDS | Mutation |
| RYR2 | 97 | 13967 | dup 13967-13972 | ins EY 4657-4658 | LQTS | Mutation |
| RYR2 | 97 | 14010 | 14010 G > C | G4671R | LQTS | Mutation |
| RYR2 | 101 | 14542 | 14542 A > G | I4848V | LQTS | Mutation |
| RYR2 | 103 | 14659 | 14659 A > G | K4887E | LQTS | Mutation |
| RYR2 | 104 | 14806 | 14806 G > A | G4936R | Gennesiance-SUDS | Mutation |
| RYR2 | 105 | 14876 | 14876 G > A | R4959Q | LQTS | Mutation |

*SUDS = sudden unexplained death syndrome
**HCM = hypertrophic cardiomyopathy.

Any appropriate method can be used to determine whether or not a mammal (e.g., human, dog, cat, horse, monkey, cow, or pig) contains a genetic mutation in an RyR2 sequence. For example, PCR can be used to amplify nucleic acid (e.g., genomic DNA or cDNA) obtained from a human. Once amplified, the nucleic acid can be sequenced and compared to wild-type RyR2 sequences to determine whether or not the nucleic acid contains a genetic mutation such as those provided herein (e.g., those provided in Table 2 or 3). In some cases, the nucleic acid sequence can be assessed to determine whether or not it encodes a mutation at an amino acid position identified on a list. Such a list can include, without limitation, one or more (e.g., two, three, four, five, six, seven, or eight) of the following amino acid positions: 186, 243, 329, 332, 357, 400, 414, 466, 919, 1724, 1837, 2113, 2267, 2387, 2392, 2420, 2475, 3800, 3938, 4124, 4196, 4307, 4556, 4565, 4657, 4658, 4887, and 4936 of SEQ ID NO:1. The list can be on a tangible medium such as a notebook page, a paper chart, or a computer medium. In some cases, the list can be stored in a database on a computer medium (e.g., a floppy or hard drive disc).

Additional examples of methods that can be used to determine whether or not a mammal contains a genetic mutation in an RyR2 sequence include denaturing high performance liquid chromatography (DHPLC; Underhill et al., Genome Res., 7:996-1005 (1997)), allele-specific hybridization (Stoneking et al., Am. J. Hum. Genet., 48:370-382 (1991) and Prince et al., Genome Res., 11 (1): 152-162 (2001)), allele-specific restriction digests, mutation specific polymerase chain reactions, single-stranded conformational polymorphism detection (Schafer et al., Nat. Biotechnol., 15:33-39 (1998)), infrared matrix-assisted laser desorption/ionization mass spectrometry (WO 99/57318), and combinations of such methods.

In some embodiments, genomic DNA can be used to detect one or more mutations in RyR2 nucleic acid. Genomic DNA typically is extracted from a biological sample such as a peripheral blood sample, but can be extracted from other biological samples, including tissues (e.g., mucosal scrapings of the lining of the mouth). Routine methods can be used to extract genomic DNA from a blood or tissue sample, including, for example, phenol extraction. In some cases, genomic DNA can be extracted with kits such as the QIAamp® Tissue Kit (Qiagen, Chatsworth, Calif.), the Wizard® Genomic DNA purification kit (Promega, Madison, Wis.), the Puregene DNA Isolation System (Gentra Systems, Minneapolis, Minn.), and the A.S.A.P.3 Genomic DNA isolation kit (Boehringer Mannheim, Indianapolis, Ind.).

A mutation in an RyR2 sequence also can be detected by analyzing an RyR2 polypeptide or a fragment thereof in a sample from the mammal (e.g., in blood or heart tissue). Any appropriate method can be used to analyze RyR2 polypeptides including, without limitation, immunological, chromatographic, and spectroscopic methods. For example, a mutation in an RyR2 sequence that results in expression of a mutant RyR2 polypeptide can be detected in a sample from a mammal using an antibody that recognizes the mutant RyR2 polypeptide but not wild-type RyR2 polypeptide. Such an antibody can, for example, recognize a mutant RyR2 polypeptide that differs from a wild-type RyR2 polypeptide by one or more amino acid residues, without recognizing a wild-type polypeptide.

An antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type, (e.g., IgG, IgM, IgD, IgA or IgY), class (e.g., IgG1, IgG4, or IgA2), or subclass. In addition, an antibody can be from any animal including birds and mammals. For example, an antibody can be a human, rabbit, sheep, or goat antibody. An antibody can be naturally occurring, recombinant, or synthetic. Antibodies can be generated and purified using any suitable methods known in the art (see, e.g., Green et al., Production of Polyclonal Antisera, in IMMUNOCHEMICAL PROTOCOLS (Manson, ed.), pages 1-5 (Humana Press 1992); Coligan et al., Production of Polyclonal Antisera in Rabbits, Rats, Mice and Hamsters, in CURRENT PROTOCOLS IN IMMUNOLOGY, section 2.4.1 (1992); Kohler & Milstein, Nature, 256:495 (1975); Coligan et al., sections 2.5.1 2.6.7; and Harlow et al., ANTIBODIES; A LABORATORY MANUAL, page 726 (Cold Spring Harbor Pub. 1988)). For example, monoclonal antibodies can be prepared using hybridoma, recombinant, or phage display technology, or a combination of such techniques. In some cases, antibody fragments can be produced synthetically or recombinantly from a gene encoding the partial antibody sequence. An anti-RyR2 polypeptide antibody can bind to RyR2 polypeptides (e.g., wild-type or mutant RyR2 polypeptides) at an affinity of at least 10⁴ mol⁻¹ (e.g., at least 10⁵, 10⁶, 10⁷, 10⁸, 10⁹, 10¹⁰, 10¹¹, or 10¹² mol⁻¹).

As described herein, the presence in a mammal (e.g., human) of one or more than one genetic mutation in an RyR2 sequence disclosed herein (e.g., one or more mutations listed in Table 1) can indicate that the mammal is susceptible to having SIDS, SUDS, syncope, a seizure, or a cardiac event. A cardiac event can be any adverse event associated with the heart including, without limitation, an exertion- or exercise-induced cardiac event, sudden cardiac death, cardiac arrest, ventricular fibrillation, ventricular tachycardia, ventricular extrasystoles, premature ventricular contractions, and ventricular bigeminy.

The presence of one or more mutations in an RyR2 sequence disclosed herein (e.g., one or more mutations set forth in Table 1) also can indicate that a mammal is more susceptible to drowning than a corresponding mammal that does not have mutations in an RyR2 sequence. In some cases, the presence in a mammal of a mutation that results in an amino acid insertion, deletion, substitution, or combination thereof at amino acid position 186, 243, 329, 332, 357, 414, 466, 919, 1724, 1837, 2387, 2420, 2475, 3800, 3938, 4124, 4196, 4307, 4556, 4657, 4658, or 4887 of SEQ ID NO:1 can indicate that the mammal is susceptible to having syncope, a seizure, or a cardiac event. In some cases, the presence in a mammal of a mutation that results in an amino acid insertion, deletion, substitution, or combination thereof at amino acid position 400, 2113, 2267, 2392, 4565, or 4936 of SEQ ID NO:1 can indicate that the mammal is susceptible to having sudden infant death syndrome or sudden unexplained death syndrome.

In some cases, the presence or absence in a mammal of one or more genetic mutations in an RyR2 sequence described herein can be used in combination with other information (e.g., results of other diagnostic tests) to determine whether or not a mammal is susceptible to drowning or having syncope, a seizure, a cardiac event, SIDS, or SUDS. For example, the presence of absence of a mutation in an RyR2 sequence can be used in combination with results of an electrocardiogram, an echocardiogram, an exercise test (e.g., an electrocardiogram treadmill exercise test or a cardiopulmonary exercise test), or a medical examination. In some cases, information about the presence or absence of a mutation in an RyR2 sequence can be used together with a medical history, a family history, or information from a Holter device that has recorded the electrical activity of the heart of a mammal over a period of time (e.g., days).

The presence in a mammal of one or more mutations in an RyR2 sequence disclosed herein (e.g., one or more mutations set forth in Table 1) also can be used to distinguish one condition (e.g., CPVT) from another condition (e.g., LQTS). For example, the presence in a mammal of one or more than one mutation in an RyR2 sequence set forth in Table 2 can indicate that the mammal has CPVT rather than LQTS, particularly when the mammal is LQTS genotype-negative. A negative LQTS genotype can be a genotype that is negative for mutations in KCNQ1/KVLQT1, KCNH2/HERG, SCN5A, KCNE1/MirK, and KCNE2/MiRP1 sequences (Tester et al., Heart Rhythm, 2(10):1099-105 (2005)). In some cases, the presence in a mammal of a mutation that results in an amino acid insertion, deletion, substitution, or combination thereof at amino acid position 466, 2387, 3800, 4124, 4556, 4657, or 4658 of SEQ ID NO:1 can indicate that the mammal has CPVT and not LQTS, particularly when the mammal is LQTS genotype-negative.

In some cases, the presence in a mammal of one or more than one mutation in an RyR2 sequence provided herein can indicate that the mammal has CPVT and not LQTS when the mammal has had exercise-induced bidirectional ventricular tachycardia, exercise-induced ventricular extrasystoles, exercise/catecholamine-induced premature ventricular contractions, or exercise-induced ventricular bigeminy, particularly when the resting QT interval is normal. In some cases, the presence in a mammal of one or more than one mutation in an RyR2 sequence can indicate that the mammal has CPVT and not LQTS when the mammal has had exertional syncope, cardiac arrest (e.g., during exertion), a near drowning, or a family history of cardiac events, typically when the resting QTc of the mammal is not prolonged (e.g., resting QTc less than or equal to about 480 ms).

Once a mammal has been identified as having CPVT or LQTS, or as being susceptible to having syncope, a seizure, a cardiac event, SIDS, or SUDS, the mammal can be treated (e.g., prophylactically) with an appropriate therapy. For example, a mammal identified as having LQTS can be treated with beta-blocker therapy, and a mammal identified as having CPVT can be treated with a calcium channel blocker and/or implantation of a defibrillator.

This document also provides methods and materials to assist medical or research professionals in determining whether or not a mammal has CPVT or LQTS, or is susceptible to having syncope, a seizure, a cardiac event, SIDS, or SUDS. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the presence or absence of a mutation in an RyR2 sequence in a sample, and (2) communicating information about the presence or absence of that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

This document also provides isolated nucleic acid molecules having a portion of an RyR2 sequence. Such nucleic acid molecules can be suspected to contain one or more of the mutations provided herein. In some cases, an isolated nucleic acid molecule can contain the sequence of an entire RyR2 exon or a portion of an RyR2 exon. For example, exon 47 can be amplified from genomic nucleic acid obtained from a human. In some cases, RT-PCR can be used to amplify RNA encoding an RyR2 polypeptide.

The term “nucleic acid” as used herein encompasses both RNA and DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

Any method can be used to obtain an isolated nucleic acid molecule provided herein including, without limitation, common molecular cloning and chemical nucleic acid synthesis techniques. For example, PCR can be used to obtain an isolated nucleic acid molecule containing an RyR2 nucleic acid sequence with a mutation set forth in Table 1.

Isolated nucleic acid molecules provided herein can be used for diagnostic purposes. For example, an isolated nucleic acid comprising a portion of an RyR2 sequence (e.g., a PCR amplicon comprising one or more than one mutation provided herein) can be used in DHPLC or allele specific hybridization analyses. An isolated nucleic acid containing a mutation also can be used in the form of a PCR primer that is about 20 nucleotides in length to amplify a region of an RyR2 sequence containing the mutation. In addition, an isolated nucleic acid containing a mutation can be labeled (e.g., with a fluorescent label) and used to detect an RyR2 sequence containing the mutation.

The invention will be further described in the following examples, which do not limit the scope of the invention described in the claims.

### EXAMPLES

### Example 1

### Identifying Mutations Associated with Increased Susceptibility to Syncope, Seizures, or Cardiac Events

### Study Participants

A comprehensive mutational analysis of all 60 amino acid encoding exons of the 5 cardiac channel LQTS-associated genes was completed for 541 consecutive, unrelated patients (358 females; average age at diagnosis was 24±16 years; average QTc=482±57 ms) who were referred to the Sudden Death Genomics Laboratory at Mayo Clinic, Rochester, Minn., for LQTS genetic testing (Tester et al., Heart Rhythm, 2:507-517 (2005)). Of those 541 patients, 269 were identified as having no LQTS-associated mutations and were referred to as “genotype-negative” LQTS. The following is an analysis of those 269 patients.

### RyR2 Mutational Analysis

A targeted mutational analysis of 23 reported CPVT1-associated exons (8, 14-15, 44-47, 49, 83, 87-91, 93-94, 97, and 100-105) in RyR2 was performed on the genotype-negative LQTS patients using polymerase chain reaction (PCR), denaturing high performance liquid chromatography (DHPLC), and DNA sequencing as described elsewhere (Priori et al., Circulation, 103:196-200 (2001); Tiso et al., Hum. Mol. Genet., 10:189-194 (2001); Priori et al., Circulation, 106:69-74 (2002); Marks, Circulation, 106(1):8-10 (2002); Lahat et al., Circulation, 107(3):e29 (2003); Laitinen et al., Circulation, 103:485-490 (2001); Bauce et al., Am. Coll. Card., 40:341-349 (2002); Bagattin et al., Clin. Chem., 50:1148-1155 (2004); and Ackerman et al., Mayo Clinic Proceedings, 78:1479-1487 (2003)). PCR primers described elsewhere were used for this study (Tiso et al., Hum. Mol. Genet., 10:189-194 (2001)).

All putative CPVT1-associated variants were denoted using known and accepted nomenclature (Antonarakis S E. Nomenclature Working Group. Recommendations for a nomenclature system for human gene mutations. Human Mutation, 11:1-3 (1998)). For example, the single letter amino acid code was used to designate non-synonymous, missense variants using the R176Q format. Here, at amino acid position 176, the ‘wild type’ amino acid (R=arginine) is replaced by glutamine (Q) on one of the alleles.

To be regarded as a CPVT1-susceptibility mutation, the variant must have involved a residue conserved across species that altered the primary amino acid structure of the encoded polypeptide in a key functional domain. Hence, synonymous single nucleotide polymorphisms were excluded from consideration. Additionally, to be considered as pathogenic, the non-synonymous variant must have been absent in 400 reference alleles (100 healthy white and 100 healthy black) obtained from the Human Genetic Cell Repository sponsored by the National Institute of General Medical Sciences and the Coriell Institute for Medical Research (Camden, N.J.).

### Statistical Analysis

Differences between continuous variables were assessed using unpaired student t-tests. Nominal variables were analyzed using chi-square analysis. A p-value<0.05 was considered statistically significant.

### Results

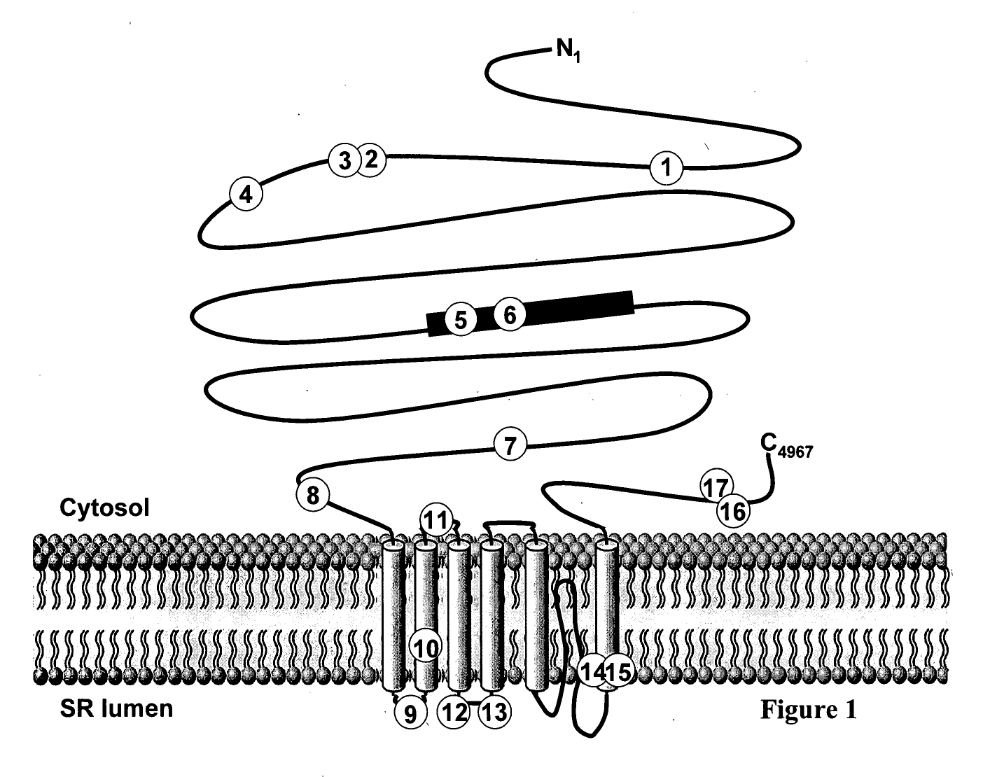

The targeted mutational analysis involving the 23 exons of the RyR2-encoded cardiac ryanodine receptor/calcium release channel identified 15 distinct CPVT1-susceptibility mutations in 17/269 (6.3%) patients (Table 2, FIG. 1). Most of the mutations were novel and missense. All mutations involved highly conserved amino acid residues and were absent in 400 reference alleles. Two mutations localized to the calstabin-2 (FKBP12.6) binding domain, while seven localized to transmembrane spanning regions (FIG. 1).

TABLE 2
CPVT1-associated RyR2 mutations in a referral cohort of LQTS

| Case | Exon | Nucleotide | Mutation | Location |
|---|---|---|---|---|
| 1 | 8 | 527 G > A | R176Q | N-term |
| 2 | 14 | 1241 G > T | R414L* | N-term |
| 3 | 14 | 1255 A > T | I419F* | N-term |
| 4 | 15 | 1396 C > G | P466A* | N-term |
| 5 | 47 | 7158 G > A | A2387T* | CBD |
| 6 | 47 | 7207 G > A | A2403T* | CBD |
| 7 | 83 | 11399 G > T | C3800F* | Cytosol |
| 8 | 90 | 12371 G > C | S4124T* | Cytosol |
| 9 | 93 | 13496 T > G | F4499C* | TM |
| 10 | 93 | 13528 G > A | A4510T* | TM |
| 11 | 94 | 13666 G > A | A4556T | TM |
| 12 | 97 | dup 13967-13972 | INS EY 4657-4658* | TM |
| 13 | 97 | 14010 G > C | G4671R* | TM |
| 14 | 101 | 14542 A > G | I4848V* | TM |
| 15 | 101 | 14542 A > G | I4848V* | TM |
| 16 | 105 | 14876 G > A | R4959Q | C-term |
| 17 | 105 | 14876 G > A | R4959Q | C-term |

*denotes a mutation unique to this patient cohort;
CBD indicates calstabin-2 (FKBP12.6) binding domain;
TM indicates transmembrane spanning domain.

The clinical phenotypes for the 17 RyR2-positive cases were summarized (Table 3). Interestingly, the referral diagnosis for all 17 RyR2 positive patients (10 females, average age at diagnosis 23±15 years) was “atypical” or “borderline” LQTS. Detailed clinical data were unavailable for two cases, which were therefore excluded from phenotype analysis. Only one individual was asymptomatic despite having a family history of sudden cardiac death (SCD), whereas 14 index cases had experienced cardiac events (syncope+cardiac arrest ˜7, syncope=5, cardiac arrest=1, seizures=1), with 11 RyR2-positive cases having multiple cardiac events.

TABLE 3
CPVT1 genotype-phenotype summary

| | Age at Dx | QTc | Exercise | | Subsequent | FH | Amino Acid |
|---|---|---|---|---|---|---|---|
| Case | (yrs)/Sex | (ms) | Stress Test | Presentation | Symptoms | SCD | Change |
| 1 | 12/M | 460 | Normal | syncope- | syncope - unknown | No | R176Q |
| | | | | excitement | trigger (4x) | | |
| 2 | 17/M | 410 | PVCs and | Near drowning | Near drowning, | No | R414L |
| | | | ventricular | | Syncope-running | | |
| | | | bigeminy | | Syncope-unknown | | |
| 3 | 51/M | 403 | NA | Asymptomatic | None | Yes (3) | I419F |
| 4 | 9/M | 460 | NA | ACA | Syncope-breath | Yes (2) | P466A |
| | | | | | holding (several) | | |
| 5 | 18/F | NA | NA | ACA | none | Yes (1) | A2387T |
| 6 | 16/F | 402 | PVCs, | Near drowning | Syncope-exertion | Yes (5) | A2403T |
| | | | ventricular | | syncope-excitement | | |
| | | | bigeminy, | | (3x) | | |
| | | | and couplets | | | | |
| 7 | 58/F | NA | NA | ACA | Syncope-startle | Yes | C3800F |
| | | | | | (several) | (NA) | |
| 8 | 14/F | 380 | NSVT (≦4 | Syncope- | Syncope-exertion | No | S4124T |
| | | | beat runs) | exertion | (several) | | |
| 9 | 34/F | 417 | NA | ACA | Syncope-anxiety (4x) | Yes (2) | F4499C |
| 10 | 11/M | 440 | PVCs, | Near drowning | Syncope-exertion | No | A4510T |
| | | | ventricular | | (4x) | | |
| | | | bigeminy, | | | | |
| | | | and couplets | | | | |
| 11 | NA/F | NA | NA | NA | NA | NA | A4556T |
| 12 | NA/M | NA | NA | NA | NA | NA | INS EY 4657-4658 |
| 13 | 10/M | 426 | PVCs, and 3 | Near drowning | Near drowning | No | G4671R |
| | | | beats of | | | | |
| | | | NSVT | | | | |
| 14 | 14/F | 429 | NA | Near drowning | Near drowning | Yes (2) | I4848V |
| 15 | 35/F | 409 | NA | Syncope | Near drowning (2x), | Yes (4) | I4848V |
| | | | | | ACA-exertion | | |
| 16 | 31/F | NA | NA | Seizure | NA | Yes (1) | R4959Q |
| 17 | 12/F | 430 | PVCs, | Syncope- | Syncope-exertion | Yes (1) | R4959Q |
| | | | ventricular | exertion | ACA-exertion | | |
| | | | bigeminy, | | | | |
| | | | and couplets | | | | |

Dx indicates diagnosis;
F indicates female;
M indicates male;
ms indicates milliseconds;
NA indicates not available;
PVCs indicates premature ventricular contractions;
NSVT indicates non-sustained ventricular tachycardia,
ACA indicates aborted cardiac arrest;
FH SCD indicates family history sudden cardiac death.
The (#) in the FH SCD column denotes the number of first- and second-degree relatives that have died suddenly.

The average QTc was 422±24 ms (range 380 to 460 ms). None of the RyR2-positive cases had a baseline QTc>480 ms. There was no reported documentation of atrial dysrhythmias, bidirectional ventricular tachycardia, or polymorphic ventricular tachycardia during exercise or catecholamine stress testing. Presumably exercise stress testing was performed in all 17 patients. However, results were provided for review in only 7 cases including 1 normal study (case 1) and exercise-induced ventricular extrasystoles in 6 cases (cases 2, 6, 8, 10, 13, and 17), which are primarily single premature ventricular contractions and ventricular bigeminy with exercise. Only 2 individuals (cases 8 and 13) had non-sustained ventricular tachycardia during stress testing.

The youngest age at which an event occurred was in a now 9 year-old male who presented to the emergency room in ventricular fibrillation as an 11 month-old infant (Table 3, case 4). There was also a family history of sudden infant death syndrome (SIDS) in this case. The majority (71%, 10/14) of the symptomatic cases experienced their cardiac events during exertion. Twelve index cases (61%) had a positive family history of cardiac events, and strikingly, 10 of these 12 cases (83%) had a history of SCD in family members less than 55 years of age. Among these 10 families, 22 family members (11 males, 9 females, and 2 gender not specified) experienced SCD with 8 (36%) being attributed to an unexplained drowning (Table 3). Nine of these 17 index cases had either a personal or family history of a drowning or near drowning. Two individuals (24-year-old male with a history of syncope and an adult female) from unrelated families experienced SCD during sleep.

The demographic and phenotypic features of the LQTS genotype-positive cases (N=272), the genotype-negative LQTS cases (N=252), and the CPVT1 genotype-positive cases (N=17) were compared (Table 4). The CPVT1 cohort had significantly lower average QTc (422 ms vs. 494 ms, p value=2.4×10⁻⁶; 422 ms vs. 471 ms, p value=0.02) and was more likely to experience syncope (80% vs. 46%, p value=0.02; 80% vs. 36%, p value=0.001) or cardiac arrest (47% vs. 13%, p value=0.003; 47% vs. 10%, p value=0.0007) than either LQTS genotype positive or genotype negative cohorts, respectively. The percentage of individuals in the CPVT1 (RyR2 positive) cohort having a family history of SCD (67%) was greater than the percentage in either the genotype positive-LQTS cohort (29%) or the genotype negative-LQTS cohort (24%), but the differences were not statistically significant (p value=0.09).

TABLE 4
Phenotypic comparison of RyR2-positive, LQTS gene-positive,
and LQTS gene-negative cohorts

| | | | | LQTS |
|---|---|---|---|---|
| | Total | RyR2 | LQTS gene | gene |
| | Cohort | positive | positive | negative |
| Number of Unrelated | 541 | 17 | 272 | 252 |
| Patients | | | | |
| Age at diagnosis | 24 ± 16 | 23 ± 15 | 23 ± 16 | 24 ± 17 |
| (range in years) | (0-78) | (0-58) | (0-75) | (0-78) |
| Sex (male/female) | 183/358 | 7/10 | 94/178 | 82/170 |
| Ethnicity (% white) | 93 | 94 | 90 | 96 |
| Average QTc (ms) | 482 ± 57 | 422 ± 24* | 494 ± 51 | 471 ± 96 |
| (range) | (365-759) | (380-460) | (402-700) | (365-759) |
| % with QTc ≧ 480 ms | 46 | 0* | 57 | 37 |
| % with syncope | 42 | 80* | 46 | 36 |
| % with cardiac arrest | 12 | 47* | 13 | 10 |
| % with positive family | 42 | 80 | 46 | 37 |
| history | | | | |
| % with positive family | 48 | 67 | 29 | 24 |
| history of SCD | | | | |
| % with Schwartz and | 29 | 0 | 41 | 17 |
| Moss score ≧ 4 | | | | |

*p-value < 0.001 in comparison to either the LQTS gene positive or gene negative subsets

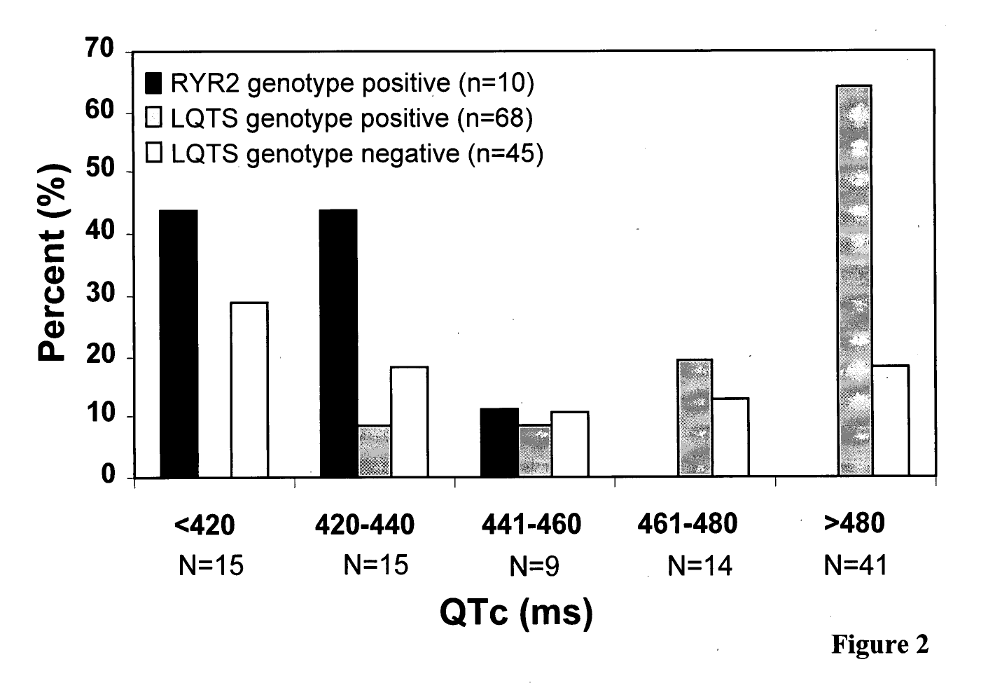

Cardiac events precipitated by exertion were common for both LQTS and CPVT. Overall, 124 (QTc available for 94) of the total original cohort of 541 unrelated LQTS referrals (23%) exhibited exertion-induced syncope or cardiac arrest with 69 (55%) being LQTS gene positive, 45 (36%) gene negative, and 10 (8%) being RyR2 gene positive (FIG. 2). However, 100% (n=9) of the RyR2 positive cases with exertional events had a QTc≦460 ms, where only 17% of LQTS gene positive cases had a QTc in this range (p value=0.3×10⁻⁶). Furthermore, none of the exertion-induced LQTS gene positive cases had a resting QTc≦420 ms (FIG. 2).

In summary, the mutations identified herein were absent in controls and localized to functionally relevant domains. Two mutations, A2387T and A2403T represent the 6th and 7th mutations reported to be localized to the calstabin-2 (FKBP 12.6) binding domain (RYR2 amino acids 2361-2496) that has been implicated in the pathogenesis of CPVT1 (Wehrens et al., Cell, 113:829-840 (2003) and Marx et al., Cell, 101:365-376 (2000)). One mutation, a duplication of nucleotides 13967-13972 resulting in the in-frame insertion of two amino acids (glutamic acid and tyrosine) between amino acid positions 4657-4658, is an insertion mutation.

The analysis is expanded to family members to establish the proportion of familial versus spontaneous germline mutations and elucidate the degree of penetrance and expressivity associated with the CPVT1-associated mutations. Based on the family histories recorded, it is estimated that the majority of the 17 cases are the result of familial mutations.

Notably, this compendium of RyR2 mutations was elicited among unrelated cases referred explicitly for LQTS genetic testing rather than a series of patients with clinically diagnosed CPVT. It appears that proper clinical recognition of and suspicion for CPVT is lagging behind LQTS. The clinical records for each RyR2-positive case posted a referral diagnosis of “atypical” or “borderline” LQTS. It has been reported that as many as 30% of their CPVT patients had been misdiagnosed as “LQTS with normal QTc interval” (Priori et al., Circulation, 106:69-74 (2002); Marks, Circulation, 106(1):8-10 (2002); and Lahat et al., Circulation, 107(3):e29 (2003)).

Phenotypic clues pointing towards a diagnosis of CPVT rather than concealed LQT1 may have been overlooked in this patient subset as well. Although exercise or catecholamine stress testing was either not performed or results were not provided for each RyR2-positive case, there was no documentation of exercise-induced bidirectional ventricular tachycardia, often considered the trademark dysrhythmia of CPVT. One patient had an entirely negative exercise stress test, while the others had exercise-induced ventricular extrasystoles usually limited to only single premature ventricular contractions or ventricular bigeminy. Such exercise/catecholamine-induced premature ventricular contractions, even if limited to exercise-induced ventricular bigeminy, can be considered as a phenotypic clue suggesting the possibility of CPVT rather than LQTS, particularly when the resting QT interval is normal. Ventricular ectopy during exercise or catecholamine stress testing in patients with genotyped LQTS is extremely uncommon (Ackerman et al., Mayo Clin. Proc., 77:413-421 (2002)).

Failure to distinguish CPVT from LQTS can prove to be a fatal mistake. Compared to the cohort of genotype positive LQTS, the unrelated cases with CPVT1 expressed a far more severe phenotype. Over half had experienced cardiac arrest. A previous study observed that one-third of CPVT cases had a family history of juvenile sudden cardiac death (Priori el al., Circulation, 106:69-74 (2002); Marks, Circulation, 106(1):8-10 (2002); and Lahat et al., Circulation, 107(3):e29 (2003)). The results provided herein indicate that 80 percent of the elucidated RyR2-positive cases had a positive family history for cardiac events including at least 22 sudden deaths among the relatives of 10 index cases. Over one-third of the sudden deaths were unexplained drownings. Through two studies involving postmortem genetic testing, RyR2 mutations were demonstrated in 2 coroner's cases of unexplained drowning and in nearly 15 percent of coroner's cases of autopsy negative sudden unexplained death (Tester er al., Mayo Clin. Proc., 80:596-600 (2005) and Tester et al., Mayo Clin. Proc., 79:1380-1384 (2004)).

Beta-blocker therapy is extremely protective for LQT1, yet such prophylactic therapy has limited success in the treatment of CPVT. It has been reported that only 41 percent of study group of CPVT patients were completely controlled by beta-blocker therapy with nearly a 4 percent annual mortality rate suggesting that such patients may be better served by a treatment of calcium channel blockers and/or implantation of an internal cardioverter-defibrillator (Sumitomo el al., Heart (British Cardiac Society), 89:66-70 (2003)). The results provided herein demonstrate that, in cases of exertional syncope, cardiac arrest during exertion, or a near drowning where the resting QTc was not prolonged, a CPVT1-associated RyR2 mutation was far more likely than concealed LQTS. The results provided herein also demonstrate that the analysis of RyR2 such as a targeted analysis of RyR2 can be used clinically as a routine diagnostic test.

### Example 2

### Identifying Mutations Associated with Increased Susceptibility to SIDS

### SIDS Cohort

Frozen necropsy tissue from 134 cases of definite or possible SIDS (57 females, 83 white, 50 black, 1 Hispanic, average age of 2.7 months; Table 5) was submitted to the Sudden Death Genomics Laboratory, Mayo Clinic, Rochester, Minn., for postmortem genetic testing. SIDS was recorded as the official cause of death on the death certificate if the autopsy, toxicology, and death scene investigation were all negative. If the autopsy was negative but a death scene evaluation either was not performed or revealed co-sleeping and the possibility of suffocation or asphyxia could not be excluded, then the cause of death was recorded as undetermined or possible SIDS. Infants whose death was due to specific disease or asphyxia were excluded.

TABLE 5
Demographics of SIDS cohort

| | SIDS Cases |
|---|---|
| Number of SIDS | 134 |
| Classification | |
| Definite SIDS | 66 |
| Possible SIDS | 68 |
| Gender | |
| Male | 77 |
| Female | 57 |
| Age at SIDS Death | |
| Mean (months) | 2.7 ± 2.0 |
| (Range) | (0.1-12) |
| Reported Ethnicity | |
| White | 83 |
| Black | 50 |
| Hispanic | 1 |

### Postmortem RyR2 Mutational Analysis

DNA was isolated from frozen tissue using the Qiagen DNeasy Tissue Kit (Qiagen, Inc, Valencia, Calif.). A targeted mutational analysis of 23 exons of RyR2 implicated in CPVT1 (exons 8, 14-15, 44-47, 49, 83, 87-91, 93-94, 97, 100-105) was performed on genomic DNA using polymerase chain reaction, denaturing high performance liquid chromatography (DHPLC), and direct DNA sequencing as described in Example 1 above.

To be regarded as a putative SIDS-causing mutation, the genetic variant was required to (i) be a non-synonymous variant (synonymous single-nucleotide polymorphisms were excluded from consideration), (ii) involve a highly conserved residue, (iii) be absent among 400 reference alleles from 100 healthy white and 100 healthy black control subjects, and (iv) result in a functionally altered, pro-arrhythmic cellular phenotype. Control genomic DNA was acquired from the Human Genetic Cell Repository sponsored by the National Institute of General Medical Sciences and the Coriell Institute for Medical Research (Camden, N.J.). Mutations were annotated using the single letter nomenclature whereby R2267H, for example, designates a DNA alteration producing a missense mutation involving a substitution of an arginine (R) by a histidine (H) at amino acid position 2267.

Two distinct and novel putative SIDS-causing mutations in RyR2 were identified in 1/50 (2%) black infants and 1/83 (1.2%) white infants (Table 6).

TABLE 6
SIDS-associated RyR2 mutations

| | | | | | Nucleotide | Amino Acid | |
|---|---|---|---|---|---|---|---|
| Gender | Race | Age | Status | Exon | Change | Change | Location |
| Female | Black | 6 months | possible- | 45 | 6800 G > A | R2267H | calstablin-2 |
| | | | SIDS | | | | binding domain |
| Female | White | 4 weeks | SIDS | 94 | 13695 C > A | S4565R | transmembrane |
| | | | | | | | spanning region |

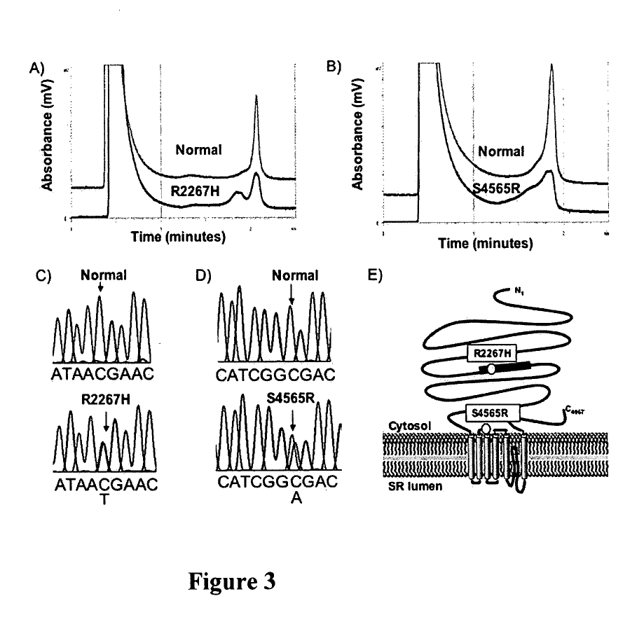

Heteroduplexes containing the R2267H or S4565R mutations were analyzing using DHPLC (FIGS. 3A and 3B, respectively). The direct DNA sequence chromatograms for R2267H and S4565R are presented in FIGS. 3C and 3D, respectively. The R2267H and S4565R mutations were also localized. The predicted linear topology of the RyR2 channel is presented in FIG. 3E, with localization of the mutations indicated by white circles.

The RyR2 missense mutations, R2267H and S4565R, were found to alter highly conserved residues. Each mutation localized to a functionally significant domain: R2267H localized near the calstabin-2 binding domain, and S4565R localized to the transmembrane spanning region. The mutations were absent in 400 reference alleles from 100 healthy white and 100 healthy black subjects.

### Effect of the R2267H Mutation on RyR2 Function

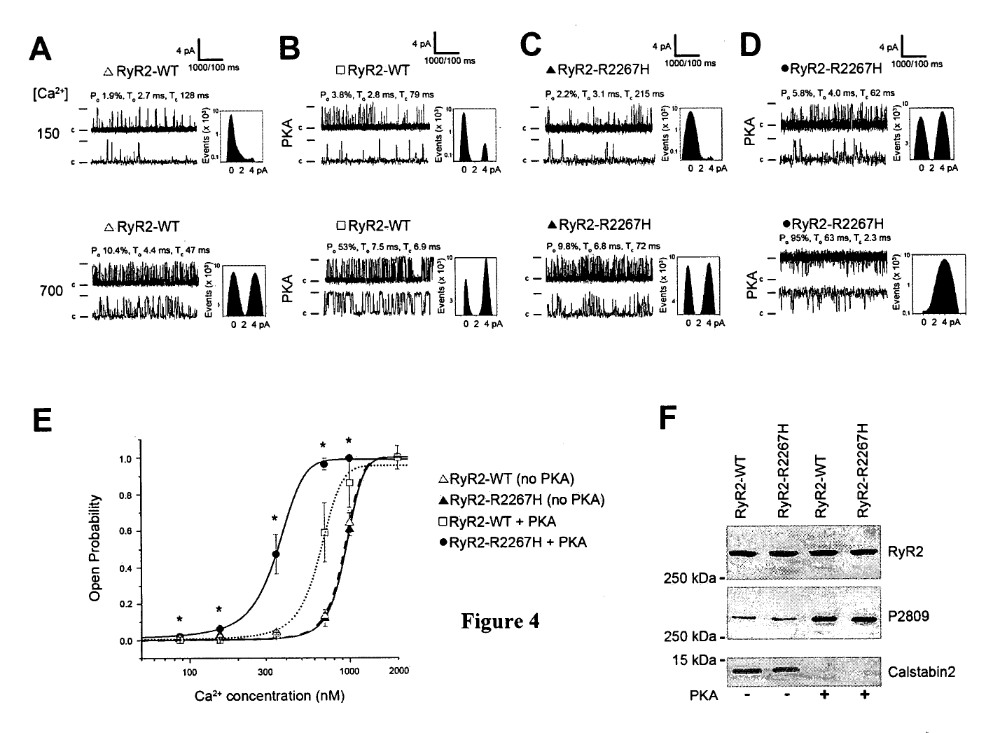

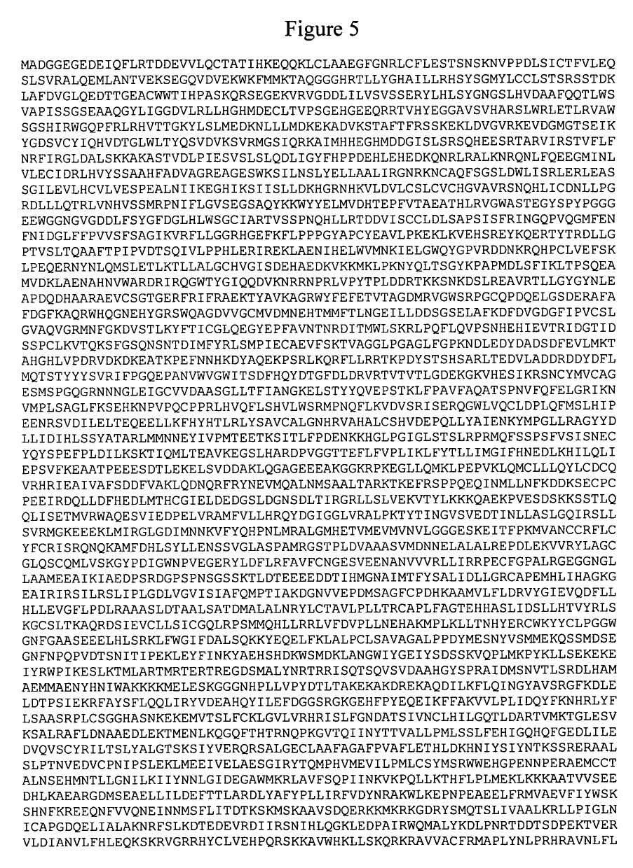

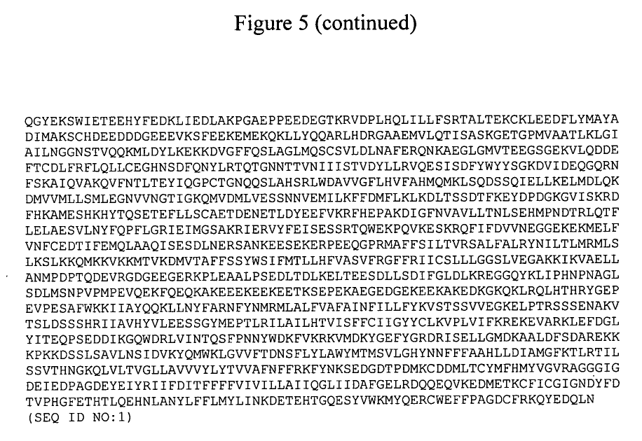

The effect of the SIDS-associated RyR2-R2267H mutation on the function of the channel was determined. Microsomes from HEK293 cells transiently coexpressing calstabin-2 and recombinant RyR2-WT (control) or RyR2-R2267H (FIG. 4F) were fused into a planar lipid bilayer, and the activity of single RyR2 channels was recorded (FIGS. 4A-4D).

To generate a hRyR2-R2267H mutant, hRyR2 cDNA cloned into a pCMV5 vector was mutated using the QuickChange II XL Site-Directed Mutagenesis Kit (Stratagene). To express homotetrameric channels, hRyR2-R2267H and hRyR2-WT, 10 μg of mutant hRyR2 cDNA or wild-type DNA were used to co-transfect HEK293 cells with 2.5 μg of calstabin-2 cDNA using the calcium phosphate precipitation method. The HEK293 cells were grown in MEM supplemented with 10% (vol/vol) FBS (Invitrogen), penicillin (100 U/mL), streptomycin (100 mg/mL), and L-glutamine (2 mmol/L).

Microsomes from HEK293 cells co-expressing calstabin-2 and RyR2-WT or RyR2-R2267H mutant were prepared as described elsewhere (Gaburjakova et al., J. Biol. Chem., 276(20):16931-5 (2001)). Briefly, the cell pellet was resuspended on ice in homogenization buffer A (10 mM HEPES, pH 7.4) supplemented with a protease inhibitor cocktail to allow for hypotonic lysis and then homogenized using a Teflon-glass homogenizer. After a second homogenization step with the addition of an equal amount of buffer B (0.5 M sucrose, 10 mM HEPES, pH 7.4), the cell lysate was centrifuged at 1,000 g for 10 minutes. Consecutive supernatants were centrifuged at 10,000 g for 15 minutes. and 100,000 g for 45 minutes. (Sorvall RC M120EX, rotor S100AT5). The pellet from the last centrifugation was resuspended in buffer C (0.25 mM sucrose, 10 mM HEPES, pH 7.4), frozen in liquid nitrogen and stored at −80° C. For the PKA phosphorylated channel experiments, the microsomes were phosphorylated in vitro by the catalytic subunit of PKA as described elsewhere (Marx et al., Cell, 101(4):365-76 (2000)).

Untreated and PKA phosphorylated RyR2-WT and RyR2-R2267H microsomes (50 μg) were immunoprecipitated with anti-RyR antibodies. Proteins were size fractionated by SDS PAGE, using a 6% gel for RyR2 and a 15% gel for calstabin-2. Immunoblots were developed using anti-RyR2 (5029, 1:5000 dilution), anti-calstabin (1:2000), or phospho-specific RyR (P2809-RyR2, 1:5000 dilution) antibodies diluted in 5% milk-TBS-tween).

The recombinant RyR2 channels were reconstituted by spontaneous fusion of microsomes into the planar lipid bilayer (mixture of phosphatidylethanolamine and phosphatidylserine in a 3:1 ratio; Avanti Polar Lipids, Alabaster, Ala.). Planar lipid bilayers were formed across a 200 μm aperture in a polysulfonate cup (Warner Instruments, Hamden, Conn.), which separated two bathing solutions: 1 mM EGTA, 250/125 mM HEPES/Tris, 50 mM KCl, 0.5 mM CaCl₂, pH 7.35 as the cis solution and 53 mM Ba(OH)₂, 50 mM KCl, 250 mM HEPES, pH 7.35 as the trans solution. After incorporation, RyR2 channel activity was recorded in the presence of various concentrations of free Ca²⁺, achieved by consecutive addition of CaCl₂ from a 20 mM stock solution, and was calculated with WinMaxC program (version 2.50; on the World Wide Web at stanford.edu/˜cpatton/maxc; Bers et al., Methods Cell Biol., 40:3-29 (1994)). The identity of the channel as RyR2 was confirmed at the end of each experiment by adding ryanodine (5 mM) into the cis solution.

Single channel currents were recorded at 0 mV using the Axopatch 200A patch-clamp amplifier (Axon Instruments, Molecular Devices, Sunnyvale, Calif.) in gap-free mode, filtered at 500 Hz, and digitized at 4 kHz. Data acquisition was performed using Digidata 1322A and Axoscope 9 software (Axon Instruments). The recordings were stored on a Pentium computer and analyzed using pClamp 6.0.2 (Axon Instruments) and Origin software (version 6.0; OriginLab, Northampton, Mass.). Data from the Ca²⁺-dependence experiments were fitted with the sigmoidal 3-parameters equation using SigmaPlot software (version 8.0, Systat Software, San Jose, Calif.).

Increased stimulation of the sympathetic nervous system during exercise or certain stages of infantile sleep is associated with increased levels of intracellular cAMP through the catecholaminergic activation of β-adrenergic receptors in the heart, which leads to PKA phosphorylation of RyR2. CPVT-associated RyR2 mutations lead to defective channel gating characterized by a “gain-of-function” phenotype with increased open probability of the channels after PKA phosphorylation when measured under conditions that simulate diastole (low cytosolic [Ca²⁺]), the resting phase of the heart cycle (Wehrens et al., Cell, 113:829-840 (2003)). These RyR2 mutations confer a leaky phenotype on the channel that leads to aberrant diastolic Ca²⁺ release from the sarcoplasmic reticulum (SR), which can initiate delayed after depolarizations (DADs), triggering ventricular arrhythmias and sudden cardiac death (Wehrens et al., Cell, 113:829-840 (2003)).

To determine whether the SIDS-linked RyR2 mutations have a similar gain-of-function, leaky phenotype, the activity of recombinant wild-type and mutant channels was measured over a range of physiological Ca²⁺ concentrations, starting at 90 nM up to full activation of the channel at about 1-5 μM Ca²⁺. Under basal conditions, the activity of the RyR2-WT channel was similar to the activity of the mutant RyR2-R2267H channel at all Ca²⁺ concentrations tested (e.g. Po of 0.019/0.104 for RyR2-WT versus Po of 0.022/0.098 for RyR2-R2267H at 150/700 nM Ca²⁺; FIGS. 4A and 4C), consistent with the clinical observation that cardiac arrhythmias in CPVT are induced only by physical exertion or emotional stress. The activity of the RyR2-WT channel was also similar to the activity of the mutant RyR2-S4565R channel over the range of Ca²⁺ concentrations under basal conditions.

To examine the functional properties of SIDS-associated RyR2 mutations under conditions simulating stress due to increased activity of the sympathetic nervous system, the activity of channels from microsomes that had been PKA phosphorylated in vitro was recorded. As illustrated by the channel activity in FIG. 4D, PKA phosphorylated SIDS mutant channels exhibited significantly higher open probabilities (Po of 0.058/0.95 for RyR2-R2267H at 150/700 nM Ca²⁺) over the physiologic range of Ca²⁺ concentrations in comparison to PKA phosphorylated WT channels (FIG. 4B, e.g. Po of 0.038/0.53 at 150/700 nM Ca²⁺, p<0.05). Similar data were obtained for the SIDS-linked mutant channel RyR2-S4565R.

Under conditions that simulate stress, such as PKA phosphorylation, during diastole (low activating [Ca²⁺]), SIDS-associated RyR2 mutant channels displayed a significant gain-of-function phenotype characterized by a leftward shift of the half-maximal activating (stimulatory) Ca²⁺ concentration (EC₅₀ of 354±40 nM for PKA phosphorylated RyR2-R2267H, n=3, versus EC₅₀ of 654±57 nM for PKA phosphorylated RyR2-WT, n=4, p<0.05, FIG. 4E). These results are consistent with the functional effect of previously characterized CPVT-associated RyR2 mutations (Wehrens et al., Cell, 113:829-840 (2003)). This gain-of-function effect was not observed for channels that were not treated with PKA (EC₅₀ of 927±19 nM for RyR2-R2267H, n=7, versus EC₅₀ of 946±23 nM for RyR2-WT, n=4, not significantly different at the 0.05 level, FIG. 4E). These results support the clinical observation that patients carrying CPVT and SIDS-linked RyR2 mutations don't die suddenly unless exposed to physical exercise, emotional stress or other factors associated with increased sympathetic activity.

The results provided herein identify functionally significant mutations in RyR2 that are linked to SIDS and that are only manifested during stress. The results provided herein also demonstrate the potential for preventing SIDS in RyR2 mutation carriers by treatment with the 1,4-benzothiazepine derivative JTV-519, which prevents RyR2-leak dependent arrhythmias in animal models (Wehrens et al., Science, 304:292-296 (2004)).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining whether or not a human contains a genetic mutation associated with an increased susceptibility of having syncope, a seizure, or a cardiac event, wherein said method comprises determining whether or not nucleic acid from said human encodes a mutation at an amino acid position of SEQ ID NO:1 identified on a list, wherein said list is on a tangible medium and comprises amino acid position 186, 243, 329, 332, 357, 414, 466, 919, 1724, 1837, 2387, 2420, 2475, 3800, 3938, 4124, 4196, 4307, 4556, 4657, 4658, or 4887 of SEQ ID NO:1.

2. The method of claim 1, wherein said cardiac event is sudden cardiac death.

3. The method of claim 1, wherein said nucleic acid comprises an exon of a human RyR2 gene.

4. The method of claim 1, wherein said nucleic acid comprises two or more exons of a human RyR2 gene.

5. The method of claim 1, wherein said list identifies a single amino acid position of SEQ ID NO:1.

6. The method of claim 1, wherein said list comprises at least two amino acid positions selected from the group consisting of amino acid positions 186, 243, 329, 332, 357, 414, 466, 919, 1724, 1837, 2387, 2420, 2475, 3800, 3938, 4124, 4196, 4307, 4556, 4657, 4658, and 4887 of SEQ ID NO:1.

7. The method of claim 1, wherein said list comprises amino acid positions 186, 243, 329, 332, 357, 414, 466, 919, 1724, 1837, 2387, 2420, 2475, 3800, 3938, 4124, 4196, 4307, 4556, 4657, 4658, and 4887 of SEQ ID NO:1.

8. The method of claim 1, wherein said tangible medium comprises a paper medium.

9. The method of claim 1, wherein said tangible medium comprises a computer medium.

10. The method of claim 1, wherein said tangible medium comprises a computer medium, and wherein said list is contained within a database stored on said computer medium.

11. The method of claim 1, wherein said method comprises reporting the presence or absence of said mutation to a medical professional or to said human.

12. The method of claim 1, wherein said method comprises classifying said human as having an increased susceptibility of having syncope, a seizure, or a cardiac event based on the presence of said mutation.

13. The method of claim 1, wherein said method comprises classifying said human as having type 1 catecholaminergic polymorphic ventricular tachycardia.

14. The method of claim 1, wherein said method comprises classifying said human as having long QT syndrome.

15. The method of claim 1, wherein said human experienced a cardiac event prior to said determining step.

16. The method of claim 1, wherein said human experienced a drowning event prior to said determining step.

17. The method of claim 1, wherein said method is performed postmortem.

18. A method for determining whether or not a human contains a genetic mutation associated with an increased susceptibility of having sudden infant death syndrome or sudden unexplained death syndrome, wherein said method comprises determining whether or not nucleic acid from said human encodes a mutation at an amino acid position of SEQ ID NO:1 identified on a list, wherein said list is on a tangible medium and comprises amino acid position 400, 2113, 2267, 2392, 4565, or 4936 of SEQ ID NO:1.

19. The method of claim 18, wherein said nucleic acid comprises an exon of a human RyR2 gene.

20. The method of claim 18, wherein said nucleic acid comprises two or more exons of a human RyR2 gene.

21. The method of claim 18, wherein said list identifies a single amino acid position of SEQ ID NO:1.

22. The method of claim 18, wherein said list comprises at least two amino acid positions selected from the group consisting of amino acid positions 400, 2113, 2267, 2392, 4565, or 4936 of SEQ ID NO:1.

23. The method of claim 18, wherein said tangible medium comprises a paper medium.

24. The method of claim 18, wherein said tangible medium comprises a computer medium.

25. The method of claim 18, wherein said tangible medium comprises a computer medium, and wherein said list is contained within a database stored on said computer medium.

26. The method of claim 18, wherein said method comprises reporting the presence or absence of said mutation to a medical professional or to said human.

27. The method of claim 18, wherein said method comprises classifying said human as having an increased susceptibility of having sudden infant death syndrome or sudden unexplained death syndrome based on the presence of said mutation.

28. The method of claim 18, wherein said human experienced increased sympathetic activity prior to said determining step.

29. The method of claim 18, wherein said method is performed postmortem.

30. An isolated nucleic acid molecule comprising a nucleic acid sequence encoding a portion of the amino acid sequence set forth in SEQ ID NO:1, wherein said nucleic acid molecule comprises a sequence that encodes an amino acid mutation at position 186, 243, 329, 332, 357, 400, 414, 466, 919, 1724, 1837, 2113, 2267, 2387, 2392, 2420, 2475, 3800, 3938, 4124, 4196, 4307, 4556, 4565, 4657, 4658, 4887, or 4936 of SEQ ID NO:1.

31. The isolated nucleic acid molecule of claim 30, wherein said portion comprises at least five contiguous amino acid residues set forth in SEQ ID NO:1.

32. The isolated nucleic acid molecule of claim 30, wherein said portion comprises at least ten contiguous amino acid residues set forth in SEQ ID NO:1.

33. The isolated nucleic acid molecule of claim 30, wherein said nucleic acid molecule comprises said portion followed by said sequence followed by a second portion of the amino acid sequence set forth in SEQ ID NO:1, wherein said portion and said second portion each comprise at least five contiguous amino acid residues set forth in SEQ ID NO:1.


SEQUENCE LISTING (1 sequence)

SEQ ID NO: 1 - protein, 4967 aa, Homo sapiens
MADGGEGEDEIQFLRTDDEVVLQCTATIHKEQQKLCLAAEGFGNRLCFLESTSNSKNVPPDLSICTFVLEQSLSVRALQEMLANTVEKSEGQVDVEKWKFMMKTAQGGGHRTLLYGHAILLRHSYSGMYLCCLSTSRSSTDKLAFDVGLQEDTTGEACWWTIHPASKQRSEGEKVRVGDDLILVSVSSERYLHLSYGNGSLHVDAAFQQTLWSVAPISSGSEAAQGYLIGGDVLRLLHGHMDECLTVPSGEHGEEQRRTVHYEGGAVSVHARSLWRLETLRVAWSGSHIRWGQPFRLRHVTTGKYLSLMEDKNLLLMDKEKADVKSTAFTFRSSKEKLDVGVRKEVDGMGTSEIKYGDSVCYIQHVDTGLWLTYQSVDVKSVRMGSIQRKAIMHHEGHMDDGISLSRSQHEESRTARVIRSTVFLFNRFIRGLDALSKKAKASTVDLPIESVSLSLQDLIGYFHPPDEHLEHEDKQNRLRALKNRQNLFQEEGMINLVLECIDRLHVYSSAAHFADVAGREAGESWKSILNSLYELLAALIRGNRKNCAQFSGSLDWLISRLERLEASSGILEVLHCVLVESPEALNIIKEGHIKSIISLLDKHGRNHKVLDVLCSLCVCHGVAVRSNQHLICDNLLPGRDLLLQTRLVNHVSSMRPNIFLGVSEGSAQYKKWYYELMVDHTEPFVTAEATHLRVGWASTEGYSPYPGGGEEWGGNGVGDDLFSYGFDGLHLWSGCIARTVSSPNQHLLRTDDVISCCLDLSAPSISFRINGQPVQGMFENFNIDGLFFPVVSFSAGIKVRFLLGGRHGEFKFLPPPGYAPCYEAVLPKEKLKVEHSREYKQERTYTRDLLGPTVSLTQAAFTPIPVDTSQIVLPPHLERIREKLAENIHELWVMNKIELGWQYGPVRDDNKRQHPCLVEFSKLPEQERNYNLQMSLETLKTLLALGCHVGISDEHAEDKVKKMKLPKNYQLTSGYKPAPMDLSFIKLTPSQEAMVDKLAENAHNVWARDRIRQGWTYGIQQDVKNRRNPRLVPYTPLDDRTKKSNKDSLREAVRTLLGYGYNLEAPDQDHAARAEVCSGTGERFRIFRAEKTYAVKAGRWYFEFETVTAGDMRVGWSRPGCQPDQELGSDERAFAFDGFKAQRWHQGNEHYGRSWQAGDVVGCMVDMNEHTMMFTLNGEILLDDSGSELAFKDFDVGDGFIPVCSLGVAQVGRMNFGKDVSTLKYFTICGLQEGYEPFAVNTNRDITMWLSKRLPQFLQVPSNHEHIEVTRIDGTIDSSPCLKVTQKSFGSQNSNTDIMFYRLSMPIECAEVFSKTVAGGLPGAGLFGPKNDLEDYDADSDFEVLMKTAHGHLVPDRVDKDKEATKPEFNNHKDYAQEKPSRLKQRFLLRRTKPDYSTSHSARLTEDVLADDRDDYDFLMQTSTYYYSVRIFPGQEPANVWVGWITSDFHQYDTGFDLDRVRTVTVTLGDEKGKVHESIKRSNCYMVCAGESMSPGQGRNNNGLEIGCVVDAASGLLTFIANGKELSTYYQVEPSTKLFPAVFAQATSPNVFQFELGRIKNVMPLSAGLFKSEHKNPVPQCPPRLHVQFLSHVLWSRMPNQFLKVDVSRISERQGWLVQCLDPLQFMSLHIPEENRSVDILELTEQEELLKFHYHTLRLYSAVCALGNHRVAHALCSHVDEPQLLYAIENKYMPGLLRAGYYDLLIDIHLSSYATARLMMNNEYIVPMTEETKSITLFPDENKKHGLPGIGLSTSLRPRMQFSSPSFVSISNECYQYSPEFPLDILKSKTIQMLTEAVKEGSLHARDPVGGTTEFLFVPLIKLFYTLLIMGIFHNEDLKHILQLIEPSVFKEAATPEEESDTLEKELSVDDAKLQGAGEEEAKGGKRPKEGLLQMKLPEPVKLQMCLLLQYLCDCQVRHRIEAIVAFSDDFVAKLQDNQRFRYNEVMQALNMSAALTARKTKEFRSPPQEQINMLLNFKDDKSECPCPEEIRDQLLDFHEDLMTHCGIELDEDGSLDGNSDLTIRGRLLSLVEKVTYLKKKQAEKPVESDSKKSSTLQQLISETMVRWAQESVIEDPELVRAMFVLLHRQYDGIGGLVRALPKTYTINGVSVEDTINLLASLGQIRSLLSVRMGKEEEKLMIRGLGDIMNNKVFYQHPNLMRALGMHETVMEVMVNVLGGGESKEITFPKMVANCCRFLCYFCRISRQNQKAMFDHLSYLLENSSVGLASPAMRGSTPLDVAAASVMDNNELALALREPDLEKVVRYLAGCGLQSCQMLVSKGYPDIGWNPVEGERYLDFLRFAVFCNGESVEENANVVVRLLIRRPECFGPALRGEGGNGLLAAMEEAIKIAEDPSRDGPSPNSGSSKTLDTEEEEDDTIHMGNAIMTFYSALIDLLGRCAPEMHLIHAGKGEAIRIRSILRSLIPLGDLVGVISIAFQMPTIAKDGNVVEPDMSAGFCPDHKAAMVLFLDRVYGIEVQDFLLHLLEVGFLPDLRAAASLDTAALSATDMALALNRYLCTAVLPLLTRCAPLFAGTEHHASLIDSLLHTVYRLSKGCSLTKAQRDSIEVCLLSICGQLRPSMMQHLLRRLVFDVPLLNEHAKMPLKLLTNHYERCWKYYCLPGGWGNFGAASEEELHLSRKLFWGIFDALSQKKYEQELFKLALPCLSAVAGALPPDYMESNYVSMMEKQSSMDSEGNFNPQPVDTSNITIPEKLEYFINKYAEHSHDKWSMDKLANGWIYGEIYSDSSKVQPLMKPYKLLSEKEKEIYRWPIKESLKTMLARTMRTERTREGDSMALYNRTRRISQTSQVSVDAAHGYSPRAIDMSNVTLSRDLHAMAEMMAENYHNIWAKKKKMELESKGGGNHPLLVPYDTLTAKEKAKDREKAQDILKFLQINGYAVSRGFKDLELDTPSIEKRFAYSFLQQLIRYVDEAHQYILEFDGGSRGKGEHFPYEQEIKFFAKVVLPLIDQYFKNHRLYFLSAASRPLCSGGHASNKEKEMVTSLFCKLGVLVRHRISLFGNDATSIVNCLHILGQTLDARTVMKTGLESVKSALRAFLDNAAEDLEKTMENLKQGQFTHTRNQPKGVTQIINYTTVALLPMLSSLFEHIGQHQFGEDLILEDVQVSCYRILTSLYALGTSKSIYVERQRSALGECLAAFAGAFPVAFLETHLDKHNIYSIYNTKSSRERAALSLPTNVEDVCPNIPSLEKLMEEIVELAESGIRYTQMPHVMEVILPMLCSYMSRWWEHGPENNPERAEMCCTALNSEHMNTLLGNILKIIYNNLGIDEGAWMKRLAVFSQPIINKVKPQLLKTHFLPLMEKLKKKAATVVSEEDHLKAEARGDMSEAELLILDEFTTLARDLYAFYPLLIRFVDYNRAKWLKEPNPEAEELFRMVAEVFIYWSKSHNFKREEQNFVVQNEINNMSFLITDTKSKMSKAAVSDQERKKMKRKGDRYSMQTSLIVAALKRLLPIGLNICAPGDQELIALAKNRFSLKDTEDEVRDIIRSNIHLQGKLEDPAIRWQMALYKDLPNRTDDTSDPEKTVERVLDIANVLFHLEQKSKRVGRRHYCLVEHPQRSKKAVWHKLLSKQRKRAVVACFRMAPLYNLPRHRAVNLFLQGYEKSWIETEEHYFEDKLIEDLAKPGAEPPEEDEGTKRVDPLHQLILLFSRTALTEKCKLEEDFLYMAYADIMAKSCHDEEDDDGEEEVKSFEEKEMEKQKLLYQQARLHDRGAAEMVLQTISASKGETGPMVAATLKLGIAILNGGNSTVQQKMLDYLKEKKDVGFFQSLAGLMQSCSVLDLNAFERQNKAEGLGMVTEEGSGEKVLQDDEFTCDLFRFLQLLCEGHNSDFQNYLRTQTGNNTTVNIIISTVDYLLRVQESISDFYWYYSGKDVIDEQGQRNFSKAIQVAKQVFNTLTEYIQGPCTGNQQSLAHSRLWDAVVGFLHVFAHMQMKLSQDSSQIELLKELMDLQKDMVVMLLSMLEGNVVNGTIGKQMVDMLVESSNNVEMILKFFDMFLKLKDLTSSDTFKEYDPDGKGVISKRDFHKAMESHKHYTQSETEFLLSCAETDENETLDYEEFVKRFHEPAKDIGFNVAVLLTNLSEHMPNDTRLQTFLELAESVLNYFQPFLGRIEIMGSAKRIERVYFEISESSRTQWEKPQVKESKRQFIFDVVNEGGEKEKMELFVNFCEDTIFEMQLAAQISESDLNERSANKEESEKERPEEQGPRMAFFSILTVRSALFALRYNILTLMRMLSLKSLKKQMKKVKKMTVKDMVTAFFSSYWSIFMTLLHFVASVFRGFFRIICSLLLGGSLVEGAKKIKVAELLANMPDPTQDEVRGDGEEGERKPLEAALPSEDLTDLKELTEESDLLSDIFGLDLKREGGQYKLIPHNPNAGLSDLMSNPVPMPEVQEKFQEQKAKEEEKEEKEETKSEPEKAEGEDGEKEEKAKEDKGKQKLRQLHTHRYGEPEVPESAFWKKIIAYQQKLLNYFARNFYNMRMLALFVAFAINFILLFYKVSTSSVVEGKELPTRSSSENAKVTSLDSSSHRIIAVHYVLEESSGYMEPTLRILAILHTVISFFCIIGYYCLKVPLVIFKREKEVARKLEFDGLYITEQPSEDDIKGQWDRLVINTQSFPNNYWDKFVKRKVMDKYGEFYGRDRISELLGMDKAALDFSDAREKKKPKKDSSLSAVLNSIDVKYQMWKLGVVFTDNSFLYLAWYMTMSVLGHYNNFFFAAHLLDIAMGFKTLRTILSSVTHNGKQLVLTVGLLAVVVYLYTVVAFNFFRKFYNKSEDGDTPDMKCDDMLTCYMFHMYVGVRAGGGIGDEIEDPAGDEYEIYRIIFDITFFFFVIVILLAIIQGLIIDAFGELRDQQEQVKEDMETKCFICGIGNDYFDTVPHGFETHTLQEHNLANYLFFLMYLINKDETEHTGQESYVWKMYQERCWEFFPAGDCFRKQYEDQLN